(19) United States
(12) Patent Application Publication
(10) Pub. No.: US 2024/0335666 A1
(43) Pub. Date: Oct. 10, 2024

(54) STOCHASTIC STIMULATION SCHEDULING

(71) Applicant: ONWARD MEDICAL N.V., Eindhoven (NL)

(72) Inventor: John Murphy, Eindhoven (NL)

(21) Appl. No.: 18/297,222

(22) Filed: Apr. 7, 2023

(51) Int. Cl. A61N 1/36; A61N 1/02

(52) U.S. Cl. CPC A61N 1/36167; A61N 1/025; A61N 1/37235

(57) ABSTRACT

A stimulation scheduling system includes a control signal generator and a controller, which can form a control loop configured to generate sets of consistent pulse schedules with a controlled degree of difficulty. The control signal generator can generate a set of consistent pulse schedules using a set of corresponding stimulation parameter distributions and provide a detection parameter based on the generation of the set of consistent pulse schedules to a controller. The controller can estimate a difficulty state based on the detection parameter and update the stimulation parameter distributions based on the estimated difficulty state. The controller can model the difficulty of generating sets of consistent pulse schedules using a difficulty distribution. The difficulty state can be a parameter of the difficulty distribution. The controller can balance the difficulty of generating sets of consistent pulse schedules against the fidelity of the generated sets of pulse schedules.

### TECHNICAL FIELD

The disclosed systems and methods concern creation of stimulation pulse schedules for neuromodulation and/or neurostimulation systems. In particular, the disclosed systems and methods concern stochastic stimulation pulse schedules that avoid phase locking or pulse skipping.

### BACKGROUND

Neuromodulation and/or neurostimulation systems can address physical or neurological injuries, diseases, or conditions of a patient by providing stimulation to the patient in complicated patterns that involve multiple stimulation channels (e.g., stimulation partitures). However, the more complicated the stimulation pattern and the more stimulation channels involved, the greater the likelihood that two stimulation channels will provide simultaneous, or nearly simultaneous, stimulation. When this happens, the effects can be synergistic and unpredictable. The patient can experience pain or unpleasant sensations; and muscles, nerves, organs, or other structures of the patient can be unintentionally or excessively activated.

Conventional approaches to preventing simultaneous activation include pre-calculating stimulation schedules, delaying pulses, or skipping pulses. However, these conventional approaches present technical problems. Pre-calculating stimulation schedules can be time consuming and inflexible, and does not guarantee the existence of a suitable stimulation schedule; delaying pulses can result in phase locking; and skipping pulses can result in irregular bursts of stimulation.

### SUMMARY

The disclosed systems and methods relate to a scheduling stimulation system. The stimulation scheduling system can include a control signal generator configured to generate pulse schedules for a set of stimulation channels using stimulation parameter distributions and a controller configured to update the stimulation parameter distributions based at least in part on the difficulty in generating consistent pulse schedules. In some embodiments, the stimulation parameter distributions can be updated based at least in part on the fidelity of the generated pulse schedules.

The disclosed embodiments include a stimulation scheduling method. The method can include sampling a stimulation parameter distribution for a first stimulation channel to obtain a stimulation parameter value. The method can further include generating a first pulse schedule for the first stimulation channel using the stimulation parameter value. The method can further include comparing the first pulse schedule to a second pulse schedule for a second stimulation channel. The method can further include, based on the comparison, providing a stimulator control signal to a stimulator. The stimulator control signal can be based on the first pulse schedule. The stimulator can be configured to provide stimulation according to the stimulator control signal on the first stimulation channel.

The disclosed embodiments include a stimulation scheduling system. The system can include a control signal generator. The control signal generator can be configured to generate a set of consistent pulse schedules according to a set of corresponding stimulation parameter distributions. The control signal generator can be further configured to provide a stimulator control signal to a stimulator. The stimulator control signal can be based on the set of consistent pulse schedules. The stimulator can be configured to provide stimulation according to the stimulator control signal on a set of stimulation channels. The control signal generator can be further configured to provide at least one detection parameter based on the generation of the set of consistent pulse schedules to a controller. The controller can be configured to estimate a difficulty state based on the at least one detection parameter. The controller can be further configured to update the stimulation parameter distributions based on the estimated difficulty state.

The disclosed embodiments include a stimulation scheduling system. The system can include an implantable pulse generator. The implantable pulse generator can be configured to target, using a control signal generator and a controller, a degree of difficulty in generating consistent pulse schedules for a set of stimulation channels by updating stimulation parameter distributions for the stimulation channels, the stimulation parameter distributions used to generate the consistent pulse schedules.

It is to be understood that both the foregoing general description and the following detailed description are exemplary and explanatory only and are not restrictive of the disclosed embodiments, as claimed.

### BRIEF DESCRIPTION OF THE DRAWINGS

The drawings are not necessarily to scale or exhaustive. Instead, emphasis is generally placed upon illustrating the principles of the embodiments described herein. The accompanying drawings, which are incorporated in and constitute a part of this specification, illustrate several embodiments consistent with the disclosure and, together with the description, serve to explain the principles of the disclosure. In the drawings:

FIG. 1A depicts pulse overlap in an exemplary stimulation partiture, consistent with disclosed embodiments.

FIG. 1B depicts phase locking in an exemplary stimulation partiture, consistent with disclosed embodiments.

FIG. 1C depicts pulse skipping in an exemplary stimulation partiture, consistent with disclosed embodiments.

FIG. 2 depicts an exemplary system capable of providing stimulation to a patient, consistent with disclosed embodiments.

FIG. 3 depicts exemplary components of a pulse generation system configured to generate sets of consistent pulse schedules, consistent with disclosed embodiments.

FIG. 4 depicts an exemplary pulse generation system, consistent with disclosed embodiments.

FIG. 5A to 5F depicts stages in the generation of an exemplary stimulation partiture, consistent with disclosed embodiments.

FIG. 6A depicts an exemplary empirical difficulty distribution, consistent with disclosed embodiments.

FIG. 6B depicts exemplary empirical stimulation parameter distributions, consistent with disclosed embodiments.

FIG. 7 depicts an exemplary process of stochastic stimulation scheduling, consistent with disclosed embodiments.

### DETAILED DESCRIPTION

Reference will now be made in detail to exemplary embodiments, discussed with regards to the accompanying drawings. In some instances, the same reference numbers will be used throughout the drawings and the following description to refer to the same or like parts. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

Stimulation, as described herein, can include electrical stimulation. In some embodiments, stimulation can include optical, mechanical, or other suitable stimulation modalities.

A patient, as described herein, can be the individual to whom stimulation is provided. A user, as described herein, can be the patient, a caregiver of the patient, or a service provider (e.g., a technician; a clinician, such as a doctor, nurse, rehabilitation specialist, physical therapist, or the like; or other service provider).

A stimulation partiture, as described herein, can specify stimulation parameters for multiple stimulation channels. In some embodiments, these stimulation channels can be configured for provision to the patient as a single unit. In some embodiments, the stimulation partiture can be designed and intended to provide a particular physiological effect or effects. For example, a stimulation partiture can specify multiple channels of stimulation that correspond to different muscles of the feet, legs, and trunk. The stimulation partiture can define a sequence of stimulation times and/or stimulation amplitudes for these channels. In this example, the stimulation partiture can be designed such that, when stimulation is provided according to the stimulation partiture by a stimulator through a suitably configured arraignment of leads to a paraplegic patient, the paraplegic patient rises to a standing position from a seated position. As may be appreciated, such a positional change requires a complex combination of muscle activations.

In some embodiments, a stimulation partiture can specify parameters for conditional execution of one or more of the stimulation channels. Such conditional execution can involve sensor signals received from the patient or the body of the patient. For example, a stimulation partiture can specify multiple channels of stimulation configured to inhibit bladder contractions and increase urethral sphincter tone in response to an increase in bladder pressure detected by a sensor implanted in the bladder of a patient. In such an instance, the stimulation partiture can define the triggering conditions, as well as the stimulation response.

Stimulation parameters, as described herein, can include inter-burst interval; number of stimulation pulses in a burst; inter-pulse interval; first phase stimulation amplitude; first phase stimulation duration; first phase pulse shape, or the like. In some embodiments, stimulation parameters can include inter-phase duration and the shapes, amplitudes, durations, or the like of any subsequent phases in a stimulation pulse (e.g., second phase stimulation amplitude, second phase stimulation duration, second phase stimulation pulse shape, or the like). In some embodiments, stimulation parameters can include the choice of stimulation contacts (e.g., which electrode is used on a multi-contact electrode); stimulation polarity (e.g., monopolar, bipolar, or the like); whether the stimulation phases are charge-balanced, whether the stimulation is current or voltage controlled, or the like.

A stimulation partiture can include multiple stimulation channels. Each stimulation channel can provide stimulation pulses to the patient according to specified stimulation parameters for that stimulation channel. In some instances, these stimulation parameters can be selected to cause the stimulation pulses to achieve a desired physiological effect, while minimizing undesired side effects. For example, the stimulation parameters for a stimulation channel can be selected to evoke or enable a target muscle contraction (e.g., as part of a gait cycle), while minimizing painful or unpleasant sensations or the activation of other nearby non-target muscles or organs. The selection of stimulation parameters for one stimulation channel may be performed independently of the selection of stimulation parameters for another stimulation channel.

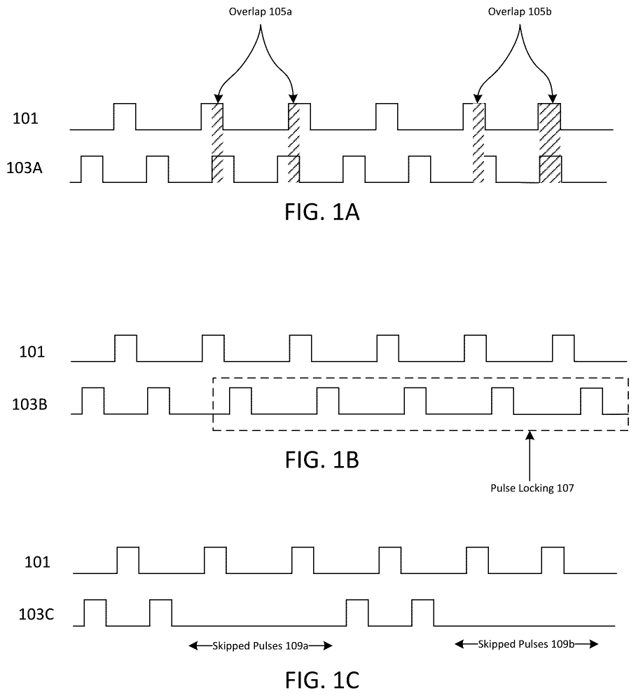

However, a stimulation partiture can specify that stimulation be provided simultaneously on multiple stimulation channels. Absent some mitigation measure, providing simultaneous stimulation on multiple stimulation channels can result in stimulation pulses of different channels overlapping (or nearly overlapping). FIG. 1A depicts two stimulation channels 101 and 103A that have differing stimulation parameters. The stimulation channels are depicted as having a “low” value that indicates no stimulation is being provided and a “high” value that indicates provision of stimulation. Overlap 105A and overlap 105b indicate overlapping stimulation pulses. In general, given two stimulation channels having differing stimulation periods p milliseconds and q milliseconds, stimulation pulses of the two stimulation channels will necessarily overlap within pq milliseconds.

When a stimulation pulses on multiple channels overlap (or nearly overlap), the two stimulation pulses can interact in unintended ways. For example, the two stimulation pulses may interact synergistically. Such synergistic interactions can have undesirable consequences, such painful or unpleasant sensations, excessive activation of target muscles or organs, or unplanned activation of other, non-target target muscles or organs.

In some instances, stimulation timings for a stimulation partiture can be pre-calculated to prevent overlapping stimulation pulses. However, such pre-calculation can be time-consuming. The difficulty of finding stimulation timings that prevent overlapping stimulation pulses can increase with the number of stimulation channels. Furthermore, changes to stimulation parameters may require re-determination of the stimulation timings. As tuning a stimulation partiture to a particular patient may require many such changes, pre-calculating stimulation timings may be clinically infeasible. Furthermore, such pre-calculation may not be able to accommodate variable stimulation patterns, such as those arising from conditional execution of stimulation channels, variable-duration stimulation, feedback control of stimulation parameters, or the on-the-fly addition, removal, enabling, disabling, or modification of stimulation parameters.

Conventional approaches to stimulation scheduling include delaying pulses and skipping pulses to prevent stimulation pulses on different channels from overlapping. But each of these techniques can create problems.

When avoiding overlapping by delaying pulses, one stimulation pulse can be delayed until another stimulation pulse ends. As depicted in FIG. 1B, this approach can result in phase locking. Stimulation channels 101 and 103B are both configured to provide stimulation pulses having a fixed inter-pulse interval. Stimulation channel 103B is configured to delay stimulation pulses that would otherwise overlap with stimulation pulses on stimulation channel 101. However, as depicted in FIG. 1B, beginning a stimulation pulse on stimulation channel 103B upon the end of a stimulation pulse on stimulation channel 101 only ensures that the next stimulation pulse on stimulation channel 103B will overlap with the next stimulation pulse on stimulation channel 101. As a result, the stimulation pulses on stimulation channel 103B become phase-locked with the stimulation pulses on stimulation channel 101 (e.g., pulse locking 107).

When avoiding overlapping by skipping pulses, one of the overlapping pulses can simply be skipped. As depicted in FIG. 1B, this approach can result in irregular stimulation, particularly when stimulation channels have similar frequencies. Stimulation channels 101 and 103C are both configured to provide stimulation pulses having a fixed inter-pulse interval. Stimulation channel 103B is configured to skip stimulation pulses that would otherwise overlap with stimulation pulses on stimulation channel 101. As a result, as stimulation channels 101 and 103C drift into and out of phase, multiple pulses in a row are skipped for stimulation channel 103C (e.g., skipped pulses 109A and 109B), converting this stimulation channel from a pattern of continuous stimulation to an irregular beat pattern.

The disclosed embodiments address these technical problems using stochastic stimulation pulse schedules. A control signal generator can sample stimulation parameter distributions to generate sampled stimulation parameter values. The control signal generator can then generate pulses schedules using the sampled stimulation parameter values. The control signal generator can test these pulse schedules are tested to determine whether they are consistent. When a set of pulse schedules is consistent, the control signal generator can provide a control signal to the stimulator. The control signal can cause the stimulator to provide stimulation in accordance with the consistent set of pulse schedules. Otherwise, the control signal generator can re-sample one or more of the stimulation parameter distributions and repeat the pulse schedule generation process.

In some embodiments, the control signal generator and a controller can maintain a control loop. The control loop can control parameters of the stimulation parameter distributions to balance the difficulty of generating consistent sets of pulse schedules against the fidelity of the generated pulse schedules. The controller can use a probability distribution to model the difficulty of generating consistent sets of pulse schedules. In some embodiments, the controller can estimate parameter(s) describing this distribution. The controller can provide updates to the stimulation parameter distributions to cause these estimated parameters to converge on specified values.

In this manner, the disclosed embodiments can accommodate stimulation partitures including many stimulation channels having differing stimulation parameters. The disclosed embodiments do not require pre-calculation of stimulation timings. The disclosed embodiments are therefore suitable for clinical use in tuning post-implantation stimulation parameters. Furthermore, the disclosed embodiments can accommodate conditional execution of stimulation channels, variable-duration stimulation, feedback control of stimulation parameters, or the on-the-fly addition, removal, enabling, disabling, or modification of stimulation parameters. Furthermore, the disclosed embodiments prevent phase locking and pulse skipping.

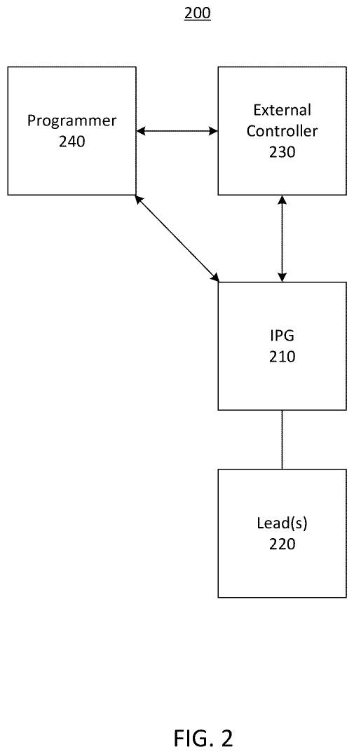

FIG. 2 depicts an exemplary system 200 capable of providing stimulation to a patient, consistent with disclosed embodiments. System 200 can enable flexible, customizable provision of stimulation to the patient. In some embodiments, programmer 240 and external controller 230 can be separate devices. Programmer 240 can be a user-facing device and be configured to enable a user to generate a stimulation partiture for a patient. Programmer 240 can therefore support creation or modification of stimulation partitures. External controller 230 can be a patient-facing device and can enable a patient or user to enable, disable, trigger, halt, or adjust stimulation provided by IPG 210 in accordance with a predetermined stimulation partiture (e.g., a stimulation partiture created by programmer 240). A programmer can be used to configure external controllers or IPGs for multiple patients, while an external controller can be associated with a particular patient.

In various embodiments, programmer 240 and external controller 230 can be implemented using the same device. For example, such a device can have multiple accounts having different privileges, such as a patient account and a user account. In such embodiments, a patient account can provide the functionality of external controller 230, while a user account can provide the functionality of programmer 240.

In various embodiments, an implantable controller can be used in place of external controller 230. The implantable controller can possess functionality of external controller 230, as described herein. In some such embodiments, the implantable controller and IPG 210 can be combined in a single device. Alternatively, the implantable controller and IPG 210 can be in separate devices.

In various embodiments, an external pulse generator can be used in place of implantable pulse generator 210. For example, an external pulse generator connected to percutaneous leads can be used to determine whether a patient is responsive to stimulation. If the patient is responsive, then the patient can be outfitted with an implantable stimulator. In such embodiments, the external pulse generator can possess functionality of implantable pulse generator 210, as described herein. Furthermore, the external pulse generator and one or more of the external controller and programmer can be implemented using a single device. For example, a single device can provide the functionality of the pulse generator and the external controller, or the pulse generator, external controller, and programmer. Alternatively, the external pulse generator, external controller, and programmer can be implemented using separate devices.

Consistent with disclosed embodiments, IPG 210 can be configured to provide stimulation signals on stimulation channels. In some embodiments, the stimulation channels can be independently controllable. IPG 210 can be configured to receive instructions from at least one of external controller 230 or programmer 240. The instructions can include instructions to load a stimulation partiture, or parameters that specify a stimulation partiture, into a memory of IPG 210. The instructions can include instructions to modify a stimulation partiture or delete a stimulation partiture from a memory of IPG 210. The instructions can include instructions to enable or disable an output of IPG 210. The instructions can include instructions to start or cease stimulation according to a stimulation partiture.

For example, IPG 210 can receive (e.g., from external controller 230 or programmer 240) a first stimulation partiture (or parameters specifying the stimulation partiture) corresponding to a component of a gait cycle. IPG 210 can provide stimulation to the patient according to the stimulation partiture. While providing stimulation according to the first stimulation partiture, IPG 210 can receive another stimulation partiture corresponding to the next component of the gait cycle. Upon completion of stimulation according to the first stimulation partiture, IPG 210 can provide stimulation according to the second stimulation partiture. As a result of the sequential execution of such stimulation partitures, the patient can be moved through a gait cycle.

In some embodiments, IPG 210 can be configured for implantation into a patient. IPG 210 can include control circuitry, communication circuitry, and a connection component. The control circuity can include at least one processor (e.g., a microprocessor, a microcontroller, an Application Specific Integrated Circuit, or another suitable processor) and at least one memory containing instructions that control the operation of IPG 210. In some embodiments, the communication circuitry can be configured to support wireless communication with external controller 230 or programmer 240. In such embodiments, the communication circuitry can include an antenna (e.g., a radio-frequency antenna, such as an antenna coil, or the like), an optical link (e.g., an optical detector, or the like), or any other suitable communication components. In some embodiments, the communication circuitry can support wired communication with external controller 230 or programmer 240. In such embodiments, communication circuitry can include suitable connectors and/or connectors for created wired percutaneous connections with other components of system 200. The connection component can enable IPG 210 to connect with leads (e.g., leads 220) to provide stimulation through the leads without unacceptable signal distortion or power loss. The disclosed embodiments are not limited to any particular interconnection architecture.

In some embodiments, IPG 210 can include a power source. In some embodiments, the power source can be configured to store power for operating IPG 210 within the device (e.g., a battery, a fuel cell, or another suitable power source). In some embodiments, the power source can be configured to obtain power from an external source (which can then be stored internally, in some embodiments). For example, IPG 210 can include a radiofrequency coil for receiving externally transmitted power.

In some embodiments, IPG 210 can include a stimulator. In some embodiments, the stimulator can be configured to convert control signals into stimulation signals that can be provided to the patient. In some embodiments, the control signals can be logic signals, an array or time series of digital data, or any other suitable control signal. For example, when the stimulation is electrical stimulation, the stimulator can be or include one or more amplifier(s). The input to the amplifier(s) can be nanowatt or microwatt control signals and the output of the amplifier can be milliwatt voltage-controlled or current controlled-stimulation signals. The stimulator can include outputs corresponding to the multiple stimulation channels of IPG 210.

In some embodiments, the components of IPG 210 can be contained inside a container. The container can be sealed (e.g., hermetically sealed) to prevent or reduce communication between the environment (e.g., the implantation site within the patient) and the interior of IPG 210. In some embodiments, the container can include an electrically conductive portion. The electrically conductive portion can serve, in some instances, as a return path for current, or as a ground reference for electrical stimulation.

Consistent with disclosed embodiments, leads 220 can be configured to provide stimulation signals to the patient. In some embodiments, leads 220 can include connectors, wiring or cabling, and stimulation contacts. Connectors can be configured to interconnect with corresponding connectors on IPG 210. Wiring or cabling can convey stimulation signals from IPG 210 to the stimulation contacts. The particular implementation of the connectors, wiring or cabling, and stimulation contacts can depend on the stimulation modality. For example, when the stimulation is electrical stimulation, the stimulation contacts can be or include electrodes (which can have contact(s)). The wiring or cabling can provide electrical connectivity from IPG 210 to the electrodes.

The disclosed embodiments are not limited to any particular electrode design. Suitable electrode designs can include array or microarray electrodes, paddle electrodes; lead electrodes including circumferential contact(s), side contact(s), or tip contact(s); cuff electrodes; wire or microwire electrodes; or any other suitable electrode design.

The disclosed embodiments are not limited to any electrodes designed for any particular purpose. Suitable electrodes include brain-computer interface electrodes, deep brain stimulation electrodes, cochlear stimulation electrodes, retinal stimulation electrodes, vagal stimulation electrodes, spinal cord stimulation electrodes, foramen stimulation electrodes, bladder or bowel control stimulation electrodes, intramuscular stimulation electrodes, efferent stimulation electrodes, afferent stimulation electrodes, autonomic stimulation electrodes, enteric stimulation electrodes, or electrodes designed for some other suitable purpose.

Consistent with disclosed embodiments, external controller 230 can be configured to enable a patient or user to enable, disable, trigger, halt, or adjust stimulation provided by IPG 210. In some embodiments, external controller 230 can extend the capabilities of IPG 210 by storing sets of stimulation partitures. In some embodiments, external controller 230 can additionally extend the capabilities of IPG 210 by storing execution conditions or relationships for stimulation partitures. External controller 230 can provide stimulation partitures to IPG 210 in response to, or in accordance with, these execution conditions or relationships. In this manner, external controller 230 can circumvent memory or computational limitations of IPG 210.

Consistent with disclosed embodiments, external controller 230 can receive instructions from programmer 240. The instructions can include instructions to load a stimulation partiture into a memory of external controller 230. The instructions can include instructions to modify or delete a stimulation partiture from a memory of external controller 230. In some embodiments, the instructions received from programmer 240 can define sets of stimulation partitures and execution conditions or relationships among these stimulation partitures.

Consistent with disclosed embodiments, external controller 230 can be configured to provide instructions to IPG 210. These instructions can include instructions to IPG 210 to load a stimulation partiture, modify or delete a stimulation partiture, enable or disable a stimulation channel of IPG 210, start or cease execution of a loaded stimulation partiture, or the like. For example, external controller 230 can receive instructions from programmer 240 that define a set of stimulation partitures corresponding to a gait cycle. The instructions can also specify an execution relationship: that these stimulation partitures be provided to IPG 210 in an appropriate sequence. Then, in response to some trigger (e.g., a patient interaction with an interface of external controller 230), external controller 230 can provide the stimulation partitures to IPG 210 in the sequence. External controller 230 can provide the stimulation partitures such that the next stimulation partiture in the gait cycle is being loaded into IPG 210, while IPG 210 is providing stimulation according to the current stimulation partiture in the gait cycle.

As an additional example, external controller 230 can receive instructions from programmer 240 that specify a stimulation partiture for mitigation of autonomic dysreflexia. The instructions can also specify an execution relationship: that this stimulation partiture be provided to IPG 210 if the blood pressure of the patient exceeds a certain threshold level.

Consistent with disclosed embodiments, external controller 230 can include a user interface. A patient can interact with the user interface to cause external controller 230 to perform certain actions. In some embodiments, these actions can depend on the instructions received by external controller 230 from programmer 240. For example, the patient can cause external controller 230 to provide stimulation partitures received from programmer 240 to IPG 210. In some embodiments, these actions can depend on the state of IPG 210. For example, the patient can cause external controller 230 to instruct IPG 210 to execute (or cease execution of) a stimulation partiture loaded onto IPG 210.

The disclosed embodiments are not limited to any particular user interface implementation. In some embodiments, the user interface can be or include a graphical user interface (e.g., a screen, touchscreen, or the like), an audio user interface, a mechanical user interface (e.g., pushbutton(s), switch(es) or the like), or other suitable user interfaces.

Consistent with disclosed embodiments, programmer 240 can be configured to enable a user to create or arrange stimulation partitures for a patient. In some embodiments, creation of a stimulation partiture can include the selection or adjustment of spatial and temporal parameters for stimulation. Such selection or adjustment can include the selection or adjustment of stimulation channels used, the mapping stimulation channels to particular electrodes in the patient, or the timing of stimulation on different stimulation channels. Such selection or adjustment can further include the selection or adjustment of stimulation parameters. In some embodiments, creation of a stimulation partiture can involve using another stimulation partiture as a template or basis. The other stimulation partiture can be a default stimulation partiture, a stimulation partiture used by another patient, or a stimulation partiture previously used by the current patient. Creation of the stimulation partiture for the current patient can include obtaining and adjusting the other stimulation partiture. The other stimulation partiture can be obtained from another device (e.g., external controller 230, IPG 210, a database, another programmer, or the like).

In some embodiments, arrangement of stimulation partitures can include the creation of execution relationships among stimulation partitures. In some instances, a user can interact with programmer 240 to create a sequence of stimulation partitures that are executed sequentially by IPG 210. In some instances, a user can interact with programmer 240 to associate conditions with the execution of the stimulation partiture. In various embodiments, programmer 240 can associate stimulation partitures with user interface elements of external controller 230 (e.g., pushbuttons or toggles), such that a patient interaction with the user interface elements can cause execution of the stimulation partiture.

As may be appreciated, programmer 240 can provide instructions configuring external controller 230. Such instructions can include stimulation partitures and execution relationships. Furthermore, in some embodiments, programmer 240 can provide instructions directly to IPG 210. These instructions can include instructions to IPG 210 to load a stimulation partiture, modify or delete a stimulation partiture, enable or disable a stimulation channel of IPG 210, start or cease execution of a loaded stimulation partiture, or the like. In this manner, a user can interact with programmer 240 to determine appropriate stimulation partitures for a patient. Once the stimulation partitures are determined, the user can transmit the determined stimulation partitures (and any execution relationships) to external controller 230. The patient (or a user) can then interact with external controller 230 to cause IPG 210 to provide the intended stimulation.

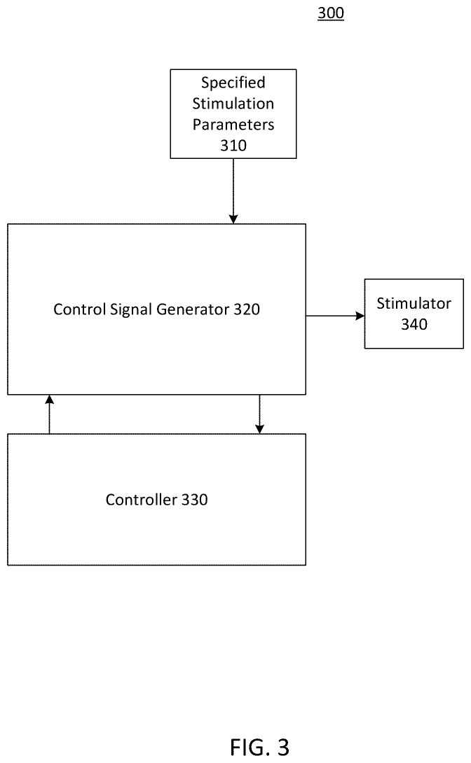

FIG. 3 depicts exemplary components of a pulse generation system 300 configured to generate sets of consistent pulse schedules, consistent with disclosed embodiments. In some embodiments, pulse generation system 300 can include a control signal generator 320, a controller 330, and a stimulator 340. Control signal generator 320 and controller 330 can form a control loop used to generate control signals for a stimulation partiture. The control signals are generated using one or more stimulation parameter distributions based on specified stimulation parameters 310. The control loop shapes the stimulation parameter distributions to reduce conflicts between control signals. The control signals can be provided to stimulator 340, which can convert the control signals into stimulation signals for provision to the patient. For convenience of description, the disclosed embodiments are described with respect to inter-pulse interval distributions. However, other stimulation parameters can be controlled according to the disclosed systems and methods to reduce conflicts between control signals. For example, pulse duration (and optionally pulse amplitude) can also be controlled to reduce conflicts between control signals. As an additional example, inter-burst interval (or the number of repeats in a burst) can also be controlled to reduce conflicts between control signals.

Consistent with disclosed embodiments, IPG 210 can be or include pulse generation system 300 (or components thereof). For example, stimulator 340 can be or include the stimulator of IPG 210 described above with regards to FIG. 2. Likewise, control signal generator 320 and controller 330 can be implemented by the control circuity of IPG 210 described above with regards to FIG. 2.

Consistent with disclosed embodiments, specified stimulation parameters 310 can concern stimulation characteristics of control signals (or equivalently stimulation channels corresponding to control signals) for a stimulation partiture. In some embodiments, the specified stimulation parameters 310 can be or include parameters defining, at least in part, stimulation parameter distributions. For convenience of description, stimulation parameter distributions are described as being normal distributions. Such distributions can be defined by a mean and a standard deviation. In some embodiments, the specified pulse parameters can be or include mean values for the stimulation parameter distributions. In some embodiments, the specified pulse parameters can include standard deviations for the stimulation parameter distributions.

However, the disclosed embodiments are not so limited. The stimulation parameter distribution need not be a normal distribution. For example, alternative distributions can include log-normal distributions, binomial distributions, f-distributions, Poisson distributions, gamma distributions, or other suitable distributions. In some embodiments, the specified pulse parameters for a distribution can be or include the expected value of the distribution (or one or more parameters that define the expected value of the distribution). In some embodiments, the specified pulse parameters for a distribution can be or include a spread describing the shape of the distribution around the expected value (e.g., a variance, standard deviation, second and/or higher order moment of the distribution, or the like).

As described herein, the specified pulse parameters can describe target values for the control signals. The stimulation characteristics of an individual pulse on a stimulation channel may not match the specified pulse parameters. However, consistent with disclosed embodiments, the average stimulation characteristics over multiple pulses on the stimulation channel can converge on the specified pulse parameters for that stimulation channel.

Consistent with disclosed embodiments, generator 320 can be configured to generate control signals for a stimulation partiture. The control signal for a stimulation partiture can be, include, or specify a pulse schedule that describes the stimulation amplitude of a stimulation channel as a function of time. A pulse schedule for a stimulation channel in a stimulation partiture can be generated by sampling a stimulation parameter distribution associated with that stimulation channel. The sampling can generate one or more stimulation parameter values. For example, when the stimulation parameter distribution describes inter-pulse intervals, the sampling can generate an inter-pulse interval value. As an additional example, when the stimulation parameter distribution describes pulse durations, the sampling can generate a pulse duration value. Generator 320 can then create a pulse schedule using the sampled values. As may be appreciated, the pulse schedule can be defined by many values, of which only a subset may be sampled. For example, a pulse schedule for a biphasic stimulation pulse may be described by an inter-pulse interval, a first phase duration, a first phase amplitude, an interphase interval, a second phase duration, and a second phase amplitude. Of these values, in this example, only the inter-pulse interval may be a sampled value. Alternatively, the inter-phase interval and a phase duration value can be sampled. In some embodiments, the remaining values can be specified as part of the stimulation partiture, received from another device (e.g., external controller 230 or programmer 240), or be default values.

Consistent with disclosed embodiments, generator 320 can construct the pulse schedule according to the sample values (and using any other relevant values). For example, when the inter-phase interval and a phase duration value are sampled, generator 320 can use these values to determine other related values of the pulse sequence. For example, the first phase duration and the second phase duration can be generated from the sampled phase duration value according to predetermined rules or formula (e.g., the phase durations may be equal, the second phase duration can be some multiple of the first phase duration, the first and second phase durations can exhibit some suitable relationship, or the like). The first phase amplitude can then be determined from the first phase duration (e.g., using a strength-duration curve, or the like). The second phase amplitude can then be determined using the first phase amplitude and the first and second phase durations (e.g., according to charge balancing principles, or the like).

Consistent with disclosed embodiments, generator 320 can determine whether the generated pulse schedule is consistent with other pulse schedules for the stimulation partiture. In some embodiments, generator 320 can compare the generated pulse schedule to other pulse schedules. The comparison can depend on one or more rules. The rules can specify temporal and/or spatial conflicts. In some embodiments, two pulse schedules can be temporally inconsistent when the two pulse schedules have temporally overlapping, or nearly overlapping pulses. A first pulse of a first pulse schedule can be overlapping with a second pulse of a second pulse schedule when the first pulse ends after the second pulse begins. In some embodiments, the first pulse can be nearly overlapping with the second pulse when the time difference between the ending time of the first pulse and the beginning time of the second pulse is less than a tolerance value (e.g., 1000 microsecond, 100 microseconds, 10 microseconds, or some other value, which may depend on the characteristics of the stimulator). In some embodiments, two pulse schedules can be spatially inconsistent when the two pulse schedules have spatially overlapping, or nearly overlapping pulses. In some embodiments, the other pulse schedules may have been created prior to this pulse schedule. In various embodiments, the other schedules may be created at the same time or created after the generated pulse schedule.

Consistent with disclosed embodiments, when the pulse schedules for the stimulation partiture are consistent, generator 320 can provide corresponding control signals to stimulator 340. In some embodiments, when a pulse schedule is inconsistent with another pulse schedule, generator 320 can recreate all of the pulse schedules. In various embodiments, only one (or both) of the inconsistent pulse schedules are recreated. After recreating one or more of the pulse schedules, generator 320 can repeat the consistency check. This cycle of consistency checking and pulse schedule recreation can be repeated until a consistent set of pulse schedules is generated.

As may be appreciated, in some instances a tradeoff may exist between the difficulty of generating consistent pulse schedules and the fidelity of the generated pulse schedules to the specified pulse parameters. As a simple example, two stimulation channels having similar inter-pulse intervals (e.g., 100 Hz and 101 Hz) may exhibit inconsistent pulse schedules for prolonged intervals (e.g., when they are in phase). Similarly, the two stimulation channels may exhibit consistent pulse schedules for prolonged intervals (e.g., when they are out of phase).

Consistent with disclosed embodiments, pulse generation system 300 can use feedback control to adapt the stimulation parameter distributions. In some embodiments, such feedback control can automatically configure generator 320 to manage the tradeoff between generating consistent pulse schedules and fidelity of the generated pulse schedules to the specified pulse parameters. To continue the prior, two-stimulation channel example, generator 320 can be configured to provide a broader spread (or even different expected value) of sampled stimulation parameter values around the specified pulse parameter values during intervals in which the two stimulation channels are inconsistent. Similarly, generator 320 can be configured to provide a narrower spread of sampled stimulation parameter values around the specified stimulation parameter values during intervals in which the two stimulation channels are consistent.

In some embodiments, generator 320 can provide detection parameter(s) to controller 330. In some embodiments, the detection parameter(s) can indicate the difficulty in generating consistent pulse schedules. In some such embodiments, the detection parameter(s) can indicate the number of attempts required to generate a consistent set of pulse schedules. In some embodiments, the detection parameter(s) can indicate the fidelity of the generated pulse schedules to the specified pulse parameters. In some such embodiments, the detection parameter(s) can indicate the differences between the sampled stimulation parameter values and the specified stimulation parameter values. For example, when the specified stimulation parameter is the inter-pulse interval, the detection parameter(s) can include a vector of differences between the sampled and specified inter-pulse intervals for the generated consistent set of pulse schedules. As may be appreciated, such a vector could likewise be generated for other stimulation characteristics. Likewise, a multi-column matrix could capture differences between sampled and specified parameter values for multiple parameters. Such a vector or matrix is referred to herein as a “jitter matrix.”

Consistent with disclosed embodiments, controller 330 can be configured to generate an update signal for the stimulation parameter distributions maintained by generator 320. The update signal can depend on the detection parameter(s) received from generator 320. In some embodiments, controller 330 can be configured to balance the ability of generator 320 to generate consistent pulse schedules and the fidelity of the generated pulse schedules. In some embodiments, controller 330 can combine a controller and an estimator or observer. The estimator or observer can be configured to determine a state of generator 320. The controller can then generate the update signal based on the determined state of generator 320.

Consistent with disclosed embodiments, the state of generator 320 can describe the present ability of generator 320 to generate consistent pulse schedules. As may be appreciated, the detection parameter(s) received from generator 320 following successful generation of a set of consistent pulse schedules may only indicate the difficulty of generating that particular set of consistent pulse schedules. The estimator or observer can use this particular sample (and optionally previously received detection parameter(s) for previously generated sets of consistent pulse schedules) to generate a general estimate of the present difficulty of generating sets of consistent pulse schedules.

In some embodiments, the difficulty of generating sets of consistent pulse schedules can be characterized by a distribution of the number of attempts required to generate a set of consistent pulse schedules. The particular number of attempts required to create the most recent consistent set of pulse schedules can be treated as a sample drawn from that distribution. The estimator or observer can estimate one or more parameters characterizing that distribution. These estimated parameters can then describe (at least in part) the state of the system.

Consistent with disclosed embodiments, the state of generator 320 can describe the fidelity of pulse schedules generated by generator 320. As may be appreciated, the detection parameter(s) received from generator 320 following successful generation of a set of consistent pulse schedules may only indicate the fidelity of that pulse schedule. The estimator or observer can use this particular sample (and optionally previously received detection parameter(s) for previously generated sets of consistent pulse schedules) to generate a general estimate of the present fidelity of pulse schedules generated by generator 320.

In some embodiments, the fidelity of pulse schedules generated by generator 320 can be characterized by at least one of error term(s) (e.g., a function or weighted function of differences between sampled and specified stimulation parameter values) or sampled stimulation parameter distribution(s). In various embodiments, the error terms or sampled stimulation parameter distributions can be per stimulation channel. In various embodiments, the error terms or sampled stimulation parameter distributions can be combined across stimulation channels. In some embodiments, the sampled stimulation parameter distributions can be probability distributions. The state of generator 320 can then include parameters describing these probability distributions. In some embodiments, the state of generator 320 can include the error term(s).

The disclosed embodiments are not limited to any particular observer or estimator architecture. In some embodiments, the observer or estimator can be a Kalman Filter (or another suitable linear or nonlinear state estimator). In some embodiments, the observer or estimator can be chosen based on an assumed type of the difficulty distribution (or sampled stimulation parameter distributions). For example, the difficulty distribution can be assumed to be an exponential distribution. An exponential distribution is characterized by a single rate parameter, which is the inverse of the expected value of the exponential function. The received detection parameter(s) can be used to generate an estimate of the single rate parameter (e.g., using an average or weighted average, such as recency-weighted average, or the like). The rate parameter can then be taken as the state of generator 320 (at least with regards to the difficulty of generating sets of consistent pulse schedules).

Consistent with disclosed embodiments, controller 330 can include a controller configured to generate an update signal based on the determined state of generator 320. The disclosed embodiments are not limited to any particular controller or control law. In some embodiments, the controller can be a proportional controller, a proportional integral controller, a proportional integral derivative controller, or the like. In some embodiments, the controller can be implemented by using a fixed mapping of states to update levels (e.g., using thresholds and if-then logic, hash coding, or another suitable implementation). In some embodiments, the controller can be implemented using a machine learning model, such as a random forest or neural network.

Consistent with disclosed embodiments, stimulator 340 can be configured to transform control signals received from generator 320 into stimulation suitable for provision to a patient. In some embodiments, stimulator 340 can be configured to receive pulse schedules and provide corresponding voltage-controlled or current-controlled electrical stimulation outputs.

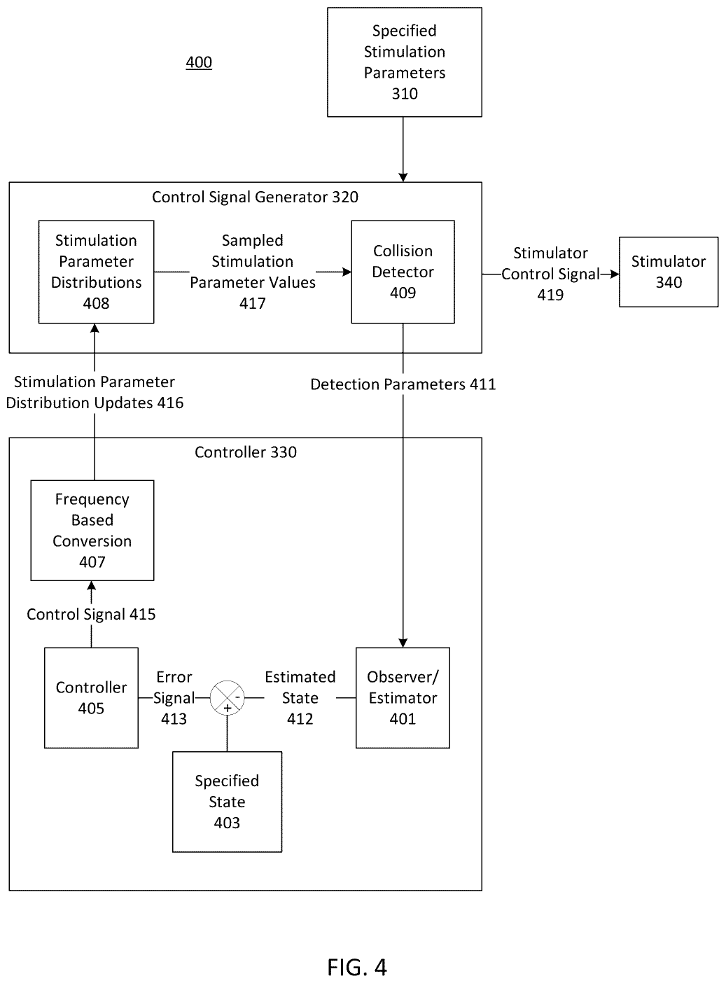

FIG. 4 depicts an exemplary pulse generation system 400, consistent with disclosed embodiments. Pulse generation system 400 can be an implementation of pulse generation system 300, described above with regards to FIG. 3. As described above with regard to FIG. 3, control signal generator 320 can be configured to produce a control signal (e.g., stimulator control signal 419). Control signal generator 320 and controller 330 can form a feedback loop in which controller 330 generates updates (e.g., stimulation parameter distribution updates 416) in response to detection parameters provided by control signal generator 320.

Consistent with disclosed embodiments, pulse generation system 400 can be configured to control inter-pulse intervals to prevent different stimulation channels from having overlapping stimulus pulses. However, as described herein, pulse generation system 400 can be configured to control other stimulation parameters. Pulse generation system 400 can be configured to model inter-pulse intervals using normal distributions. However, as described herein, pulse generation system 400 can be configured to model stimulation parameters using other probability distributions.

Consistent with disclosed embodiments, pulse generation system 400 can be configured to control the difficulty of generating sets of consistent pulse schedules. The difficulty of generating sets of consistent pulse schedules can be characterized in terms of the number of attempts required to generate a consistent pulse schedule. As may be appreciated, other measures of the difficulty of generating consistent pulse schedule may also be used.

Consistent with disclosed embodiments, pulse generation system 400 may not expressly control fidelity of the generated sets of pulse signals. Instead, pulse generation system 400 can be configured to target a particular level of difficulty in generating sets of consistent pulse schedules. In such embodiments, control of fidelity can be achieved indirectly, through the selection of the difficulty target (e.g., as fidelity and control signal generation difficulty can have inverse relationship). However, as may be appreciated, fidelity can also be controlled directly (e.g., making the control law used to generate the update dependent on a measure of fidelity).

Consistent with disclosed embodiments, observer/estimator 401 can be configured to estimate a state of control signal generator 320. Observer/estimator 401 can estimate this state using detection parameters 411. In some embodiments, detection parameters 411 can include a number of attempts required to generate the most recent set of consistent pulse schedules. In some embodiments, pulse generation system 400 can assume that the number of attempts follows a particular distribution type and estimate values for parameter(s) of that distribution. The estimated values can be, or be used to, generate the state.

In some embodiments, observer/estimator 401 can assume that the difficulty distribution is an exponential distribution. As may be appreciated, in some instances the empirical distribution may be closer to a geometric progression. However, the probability distribution for a geometric progression may be difficult to model.

Observer/estimator 401 can update an estimated expected value for the number of attempts required to generate a set of consistent pulse schedules. For example:

$${λ}_{i}^{- 1} = ( 1 - α ) {λ}_{i - 1}^{- 1} + α {n}_{i}$$

where λᵢ is the estimated rate parameter for the exponential distribution, λᵢ₋₁ is the prior estimate of the rate parameter for the exponential distribution, nᵢ is the number of attempts required to generate the current set of consistent pulse schedules, and α is a weighting parameter between 0 and 1. The larger λ, the more weight the system will place on recent numbers of attempts.

In some embodiments, estimated state 412 can be λᵢ. In such embodiments, specified state 403 can be a target rate parameter λ_{target}. Error signal 413 can then be the difference between λ_{target} and λᵢ.

In some embodiments, estimated state 412 can depend on the cumulative density function of the estimated distribution, evaluated for the most recent number of attempts. For example, assuming an exponential distribution:

$${S}_{i} = P ( N \leq {n}_{i} ; {λ}_{i} ) = 1 - {e}^{- {λ}_{i} {n}_{i}}$$

Where Sᵢ is the estimated state.

The state formula may be adjusted to account for the empirical attempt distribution being zero for values of n less than 1 (e.g., unlike a conventional exponential distribution). In some embodiments, for example, nᵢ can be replaced with:

$$x = {n}_{i} - y$$

where y is an offset.

When estimated state 412 is expressed in terms of a cumulative density function, specified state 403 can be similarly expressed. For example, assuming an exponential distribution:

$${S}_{target} = P ( N \leq {n}_{i} ; {λ}_{target} ) = 1 - {e}^{- {λ}_{target} {n}_{i}}$$

where the specified state 403 is defined in terms of a specified rate parameter λ_{target}. S_{target} can be adjusted using an offset, as described above with regards to Sᵢ. In some embodiments, error signal 413 can depend on the difference between Sᵢ and S_{target}. In various embodiments, error signal 413 can further depend on a difference between the number of attempts and an offset:

$${e}_{i} = ( {n}_{i} - y ) ( {S}_{target} - {S}_{i} )$$

where eᵢ is the error signal and y is a specified offset, as described above.

Consistent with disclosed embodiments, pulse generation system 400 can be configured to use controller 405 and frequency-based conversion 407 to generate stimulation parameter updates 416 from error signal 413.

In some embodiments, when using a control architecture in which the present output depends, at least in part, on prior error values, controller 405 can include anti-windup control. Such anti-windup control can prevent or reduce overshoot arising from sudden changes in the error signal. In some embodiments, the anti-windup control can limit or cap the contribution from prior error signal values. For example, when using integral control, the integral error term can accumulate the error signal:

$${k}_{i} = {k}_{i - 1} + {e}_{i}$$

where kᵢ is the present integral error term, kᵢ₋₁ is the prior integral error term, and eᵢ is the present error signal. The anti-windup control can cap the value of kᵢ to some predetermined value kₘₐₓ.

Consistent with disclosed embodiments, controller 405 can be configured to implement a proportional, proportional integral, or proportional integral derivative control law. For example, controller 405 can implement the following control law:

$${u}_{i} = {pe}_{i} + {d}_{i} + {k}_{i}$$

where uᵢ is the output of controller 405 (e.g., control signal 415, or the like), p is a proportional control coefficient, dᵢ is a term based on the derivative of the error signal eᵢ with respect to time, and kᵢ is the integral error term described above.

Consistent with disclosed embodiments, controller 405 can be configured to further filter the output. Such filtering can smooth the output and provide an additional degree of configurability of system 400. For example,

$${u}_{i} = α {u}_{i - 1} + ( 1 - α ) {u}_{i}$$

where uᵢ₋₁ is the prior output of controller 405 and α is a weighting coefficient between zero and one. In some embodiments, bounds can be imposed on uᵢ, depending on the physical significance of this parameter. For example, when uᵢ corresponds to a spread in a stimulation parameter distribution, a lower bound can be imposed on uᵢ (e.g., uᵢ=max(uₘᵢₙ, uᵢ), where uₘᵢₙ≥0), as a negative variance can lack physical meaning.

As may be appreciated, controller 405 is not limited to the above controller architecture. In various embodiments, controller 405 can use another, suitable controller architecture.

Consistent with disclosed embodiments, frequency-based conversion 407 can be configured to convert the control signal 415 into stimulation parameter distribution updates 416. In some embodiments, this conversion can depend on specified simulation parameters 310. For example, the stimulation parameter distribution update for a stimulation channel can be the product of a scaling factor and the control signal. The scaling factor can depend on at least one specified stimulation parameter(s) for the stimulation channel (e.g., one or more of specified simulation parameters 310).

Consistent with disclosed embodiments, the specified simulation parameters 310 can include specified inter-pulse intervals for the stimulation channels (or specified stimulation frequencies). The scaling factor for a channel can depend on the specified inter-pulse interval for the stimulation channel. In some embodiments, the specified inter-pulse interval for the stimulation channel can be normalized. The normalization factor can be the sum of the specified inter-pulse intervals for some or all of the stimulation channels:

$${p}_{ij} = {u}_{i} {μ}_{j} / {\sum }_{j} {μ}_{j}$$

where pᵢⱼ is the i^{th} update of the j^{th} stimulation channel and μⱼ is the specified mean inter-pulse interval for the j^{th} stimulation channel.

Consistent with disclosed embodiments, controller 330 can provide the stimulation parameter distribution updates 416 to control signal generator 320. As described herein, control signal generator 320 can be configured to maintain stimulation parameter distributions 408. Stimulation parameter distributions 408 can specify stimulation distribution parameters for stimulation channels.

Consistent with disclosed embodiments, a stimulation channel can be associated with a number of stimulation distribution parameters. The number of stimulation parameters associated with the stimulation channel can depend on the number of stimulation parameters sampled for that stimulation channel. The number of stimulation parameters associated with the stimulation channel can also depend on the type of distribution from which the stimulation parameters are drawn. For example, when the stimulation amplitude and inter-pulse interval are sampled for a stimulation channel, two stimulation parameter distributions can be associated with that stimulation parameter. When these two stimulation parameter distributions are modeled as normal distributions, each can have a mean and a variance. Four stimulation distribution parameters can therefore be associated with the stimulation channel (two means and two variances). Other stimulation parameter distribution model type may require fewer (e.g., exponential models) or more (e.g., Weibull models) stimulation parameters.

Consistent with disclosed embodiments, a stimulation distribution parameter can be associated with multiple stimulation channels. For example, multiple stimulation channels may draw stimulation amplitude parameters specified by the same parameters (e.g., for normal distributions, the same mean and variance, or potentially differentially scaled versions of the same mean and variance).

In some embodiments, stimulation parameter distribution updates 416 can affect a subset of the stimulation distribution parameters associated with a stimulation channel. For example, when a stimulation parameter distribution for a stimulation channel is modeled as a normal distribution, the stimulation parameter distribution updates may affect only the variance parameter of normal distribution.

In some embodiments, stimulation parameter distribution updates 416 can affect a subset of the stimulation channels. For example, stimulation parameters for one or more protected channels may not be updated by controller 330. In some embodiments, a protected channel can have predetermined stimulation parameter distribution(s). In some embodiments, all the stimulation parameters for a protected channel can be deterministic (e.g. not drawn from a distribution).

Consistent with disclosed embodiments, stimulation parameter distributions 408 can include stimulation parameters specifying an inter-pulse interval distribution for each stimulation channel. In some embodiments, these inter-pulse interval distributions can be modeled as normal distributions. The means of these normal distributions can be specified (e.g., by specified stimulation parameters 310). The variances these normal distributions can be updated using stimulation parameter distribution updates 416:

$${σ}_{ij} = {σ}_{i - 1 , j} + {p}_{ij}$$

where σᵢⱼ is the i^{th} update of the variance for the j^{th} stimulation channel, σ_{i-1,j} is the prior update of the variance for the j^{th} stimulation channel, and pᵢⱼ is the i^{th} update of the j^{th} stimulation channel. The updated stimulation parameter distributions for the j^{th} stimulation channel can then be [symbol] (μⱼ, σᵢⱼ).

Consistent with disclosed embodiments, sampled pulse parameters 418 can be drawn from the updated stimulation parameter distributions 408. Control signal generator 320 can be configured to generate pulse schedules using stimulation parameters including the sampled pulse parameters.

Consistent with disclosed embodiments, collision detector 409 can be configured to determine when pulse schedules for different stimulation channels have overlapping stimulus pulses. Such inconsistent pulse schedules can be rejected and control signal generator 320 can generate another pulse schedule using sampled pulse parameters 418.

Consistent with disclosed embodiments, control signal generator 320 can be configured to track the number of attempts required to generate a consistent set of pulse schedules. This number can be provided to controller 330 in detection parameters 411. In this manner, the control loop can be closed.

Consistent with disclosed embodiments, control signal generator 320 can provide stimulator control signal 419 to stimulator 340. Stimulator control signal 419 can specify the consistent set of pulse schedules generated by control signal generator 320 using the sampled pulse parameters 418. Stimulator 340 can generate stimulation for the patient in accordance with the stimulator control signal 419.

In some embodiments, stimulator control signal 419 can indicate only the current control signal value for a stimulation channel, even when the set of pulse schedules includes a future pulse for that stimulation channel. For example, a pulse schedule may indicate that a stimulation channel is currently not outputting a stimulation pulse, but will output a stimulation pulse in 10 ms. In this example, stimulator control signal 419 may only indicate that the stimulation channel is currently not outputting a stimulation pulse. In 10 ms, the value of stimulator control signal 419 may change, to indicate that the stimulation channel should output a stimulation pulse. Consistent with disclosed embodiments, stimulator control signal 419 may indicate only the current control signal value for a stimulation channel because the future stimulation pulse timings may change, as inconsistent pulse schedules are discarded or revised. In such embodiments, stimulator 340 can be configured to provide stimulation for the simulation channels according to the current control signal values.

In some embodiments, stimulator control signal 419 can provide information about current and future stimulation channel values. For example, a pulse schedule may indicate that a particular stimulation channel is currently not outputting a stimulation pulse, but will output a stimulation pulse in 10 ms. In this example, stimulator control signal 419 may indicate both that (i) the particular stimulation channel is currently not outputting a stimulation pulse, and (ii) the particular stimulation channel will output a stimulation pulse in 10 ms. In such embodiments, should control signal generator 320 subsequently generate a new set of pulse schedules (e.g., having a different pulse timing for the particular stimulation channel), control signal generate 320 will provide that new pulse schedule to stimulator 340. In such embodiments, stimulator 340 can be configured to determine which set of pulse schedules is applicable (e.g., the most recently received). Stimulator 340 can then provide stimulation according to that set of pulse schedules.

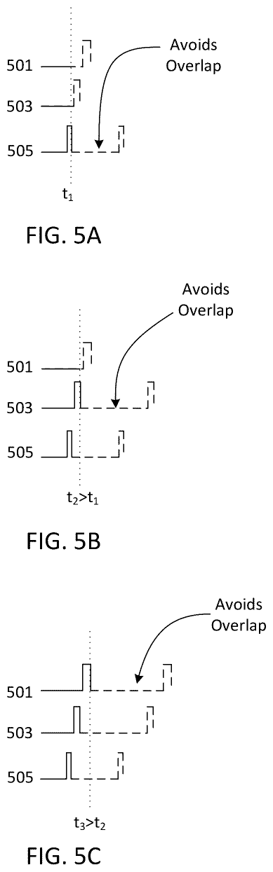

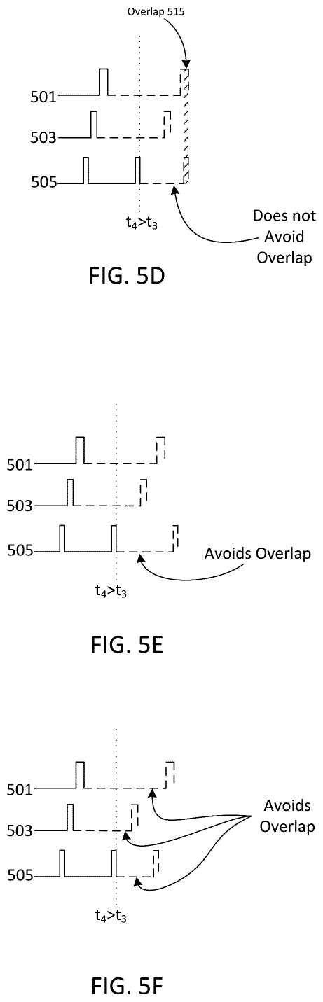

FIG. 5A to 5F depicts stages in the generation of an exemplary stimulation partiture, consistent with disclosed embodiments. The exemplary stimulation partiture can include three stimulation channels 501 to 505. Each of the three pulse schedules depicted in FIGS. 5A to 5F can correspond to one of these stimulation channels (e.g., a first pulse schedule can correspond to stimulation channel 501, a second pulse schedule can correspond to stimulation channel 503, and a third pulse schedule can correspond to stimulation channel 505). The pulse schedules may have been generated by control signal generator (e.g., control signal generator 320) using sampled stimulation parameter values (e.g., sampled stimulation parameter values 417). In this simple example, a control signal is depicted as a time series having either the value “low” or the value “high.” The value “low” can correspond to the absence of a stimulation pulse, and the value “high” can correspond to the presence of a stimulation pulse. Solid lines indicate control signals values in the past, while broken lines depict the future pulse schedules. As may be appreciated, other control signal implementations (e.g., using absolute or relative pulse start and stop times, pulse countdown timer values, or the like) can also be used.

In the example depicted in FIG. 5A to 5F, each stimulation parameter is associated with a single stimulation parameter distribution. The stimulation parameter distribution for a stimulation channel is sampled to provide the inter-pulse interval for that stimulation channel. The other stimulation parameters for each channel are specified (e.g., in specified stimulation parameters 310, or the like) and are deterministic. The control signal generator generates pulse schedules for each stimulation channel using the combination of a sampled inter-pulse interval value and the deterministic values of the other stimulation parameters.

FIG. 5A depicts the stimulation channels and associated pulse schedules at a first time t₁. In this example, a scheduled pulse completed at time t₁ on stimulation channel 505. In response to completion of this pulse, the control signal generator can generate a new pulse schedule for stimulation channel 505 (shown as a broken line depicting the inter-pulse interval and the next pulse). The control signal generator can generate this new pulse schedule using sampled stimulation parameters for stimulation channel 505. In some embodiments, the sampled stimulation parameters can be reused from the generation of the last set of pulse schedules. In some embodiments, the control signal generator can generate new sampled stimulation parameters by sampling stimulation parameter distributions for stimulation channel 505.

In this example, control signal generator can determine that the scheduled pulse for stimulation channel 505 is consistent with the scheduled pulses for stimulation channels 501 and 503. The control signal generator can therefore output detection parameter values to the controller that indicate that a single attempt was necessary to generate a consistent set of pulse schedules.

FIG. 5B depicts the stimulation channels and associated pulse schedules at a second time t₂>t₁. In this example, a scheduled pulse completed at time t₂ on stimulation channel 503. In response to completion of this pulse, the control signal generator can generate a new pulse schedule for stimulation channel 503 (shown as a broken line depicting the inter-pulse interval and the next pulse). The control signal generator can generate this new pulse schedule using sampled stimulation parameters (e.g., reused or newly generated) for stimulation channel 503.

In this example, control signal generator can determine that the scheduled pulse for stimulation channel 503 is consistent with the scheduled pulses for stimulation channels 501 and 505. The control signal generator can therefore output detection parameter values to the controller that indicate that a single attempt was necessary to generate a consistent set of pulse schedules.

FIG. 5C depicts the stimulation channels and associated pulse schedules at a third time t₃>t₂. In this example, a scheduled pulse completed at time t₃ on stimulation channel 501. In response to completion of this pulse, the control signal generator can generate a new pulse schedule for stimulation channel 501 (shown as a broken line depicting the inter-pulse interval and the next pulse). The control signal generator can generate this new pulse schedule using sampled stimulation parameters (e.g., reused or newly generated) for stimulation channel 501.

In this example, control signal generator can determine that the scheduled pulse for stimulation channel 501 is consistent with the scheduled pulses for stimulation channels 503 and 505. The control signal generator can therefore output detection parameter values to the controller that indicate that a single attempt was necessary to generate a consistent set of pulse schedules.

FIG. 5D depicts the stimulation channels and associated pulse schedules at a third time t₄>t₃. In this example, a scheduled pulse completed at time t₄ on stimulation channel 505. In response to completion of this pulse, the control signal generator can generate a new pulse schedule for stimulation channel 505 (shown as a broken line depicting the inter-pulse interval and the next pulse). The control signal generator can generate this new pulse schedule using sampled stimulation parameters (e.g., reused or newly generated) for stimulation channel 505.

In this example, control signal generator can determine that the scheduled pulse for stimulation channel 505 is inconsistent with the scheduled stimulation pulse for stimulation channel 501. The scheduled stimulation pulse on stimulation channel 501 overlaps with the scheduled stimulation pulse for stimulation channel 505, resulting in overlap 515. The control signal generator can therefore re-attempt generation of a pulse schedule for stimulation channel 505.

FIG. 5E depicts the stimulation channels and associated pulse schedules at a third time t₄>t₃, following a second attempt at generating a pulse schedule for stimulation channel 505. In this example, the control signal generator obtained a new inter-pulse interval by resampling the stimulation parameter distribution for stimulation channel 505. The new inter-pulse interval is longer than the prior inter-pulse interval. As a result, the pulse schedules for stimulation channels 501 and 505 are consistent (e.g., the stimulation pulses no longer overlap). A control signal generator can therefore output detection parameter values to the controller that indicate that two attempts were necessary to generate a consistent set of pulse schedules.

FIG. 5F depicts the stimulation channels and associated pulse schedules at a third time t₄>t₃, following a second attempt at generating a pulse schedule for stimulation channel 505. In this example, the control signal generator obtained a new inter-pulse interval by resampling the stimulation parameter distribution for all stimulation channels (e.g., as opposed to only stimulation channel 505). The new inter-pulse intervals for the stimulations differ from those of the prior stimulation channels. As a result, the pulse schedules for stimulation channels 501 and 505 are consistent (e.g., the stimulation pulses no longer overlap). A control signal generator can therefore output detection parameter values to the controller that indicate that two attempts were necessary to generate a consistent set of pulse schedules.

As may be appreciated, when all stimulation channels are updated upon detection of a conflict, an inter-pulse interval of a stimulation channel may be shortened such that the resulting pulse schedule would place the pulse in the past. The control signal generator can be configured to treat sets of pulse schedules that include pulses occurring in the past as inconsistent. In some embodiments, such an inconsistent set of pulse schedules may not contribute to the tracked number of attempts. In some embodiments, the sampled inter-pulse interval for the stimulation channel having the pulse in the past may be resampled. In some embodiments, the sampled inter-pulse intervals for all the stimulation channels may be resampled. In some embodiments, such an

In some embodiments, the control signal generator can be configured to generate a new pulse schedule upon completion of a scheduled pulse. In such embodiments, pulse schedules may be generated “as-needed” while stimulation is being provided to the patient. In some embodiments, the control signal generator can be configured to generate pulse schedules for multiple pulses. For example, the pulse generation process depicted in FIG. 5A to 5F can be performed in advance. In such embodiments, the control signal generator can be configured to simulate stimulation over a duration, generating a pulse schedule including many pulses for some or all of the stimulation channels in the stimulation partiture. This multi-pulse, multi-channel pulse schedule can then be replayed to provide the control signal for the stimulator. As may be appreciated, each time this multi-pulse, multi-channel pulse schedule is generated, the pulse timings may differ, due to the stochastic nature of the pulse-to-pulse stimulation parameters. However, the expected values for each stimulation channel will remain close to the specified stimulation parameter values for that stimulation channel.

In some embodiments, a programmer (e.g., programmer 240) or external controller (e.g., external controller 230) can be configured to generate pulse schedules for multiple pulses, according to the embodiments described herein. For example, the control signal generator and controller can be implemented by the programmer or the external controller. The multi-pulse, multi-channel pulse schedule can then be provided to an IPG (e.g., IPG 210), which can use the pulse schedule in providing stimulation to a patient.

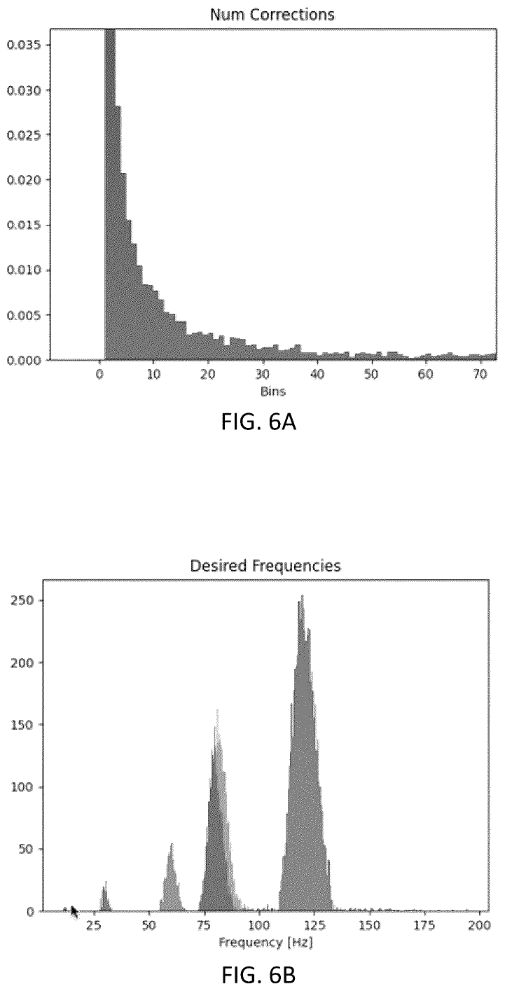

FIG. 6A depicts an exemplary empirical difficulty distribution, consistent with disclosed embodiments. In this example, the only sampled stimulation parameter was the inter-pulse interval. The stimulation parameter distribution was a normal distribution having a mean and variance. The variance was controlled using the control signal generator and the controller as described in FIGS. 3 and 4. The detection parameter was the number of attempts required to generate a set of consistent pulse schedules. The independent variable in the empirical difficulty distribution is the number of attempts, and the dependent variable is the number of times that number of attempts was required. As shown in FIG. 6A, the empirical difficulty distribution resembles an exponential distribution. As an exponential distribution can be modeled using a single rate parameter, assuming that the empirical difficulty distribution is an exponential distribution can enable state control, with the rate parameter being the controlled state.

FIG. 6B depicts exemplary empirical stimulation parameter distributions, consistent with disclosed embodiments. In this example, the only sampled stimulation parameter was the inter-pulse interval. The stimulation parameter distribution was a normal distribution having a mean and variance. The variance was controlled using the control signal generator and the controller as described in FIGS. 3 and 4. The independent variable is the reciprocal of the inter-pulse interval for each stimulation pulse output, while the dependent variable is the number of times that an inter-pulse interval falling in a binned range of inter-pulse intervals was output. In the example depicted in FIG. 6B, the stimulation partiture included six stimulation channels. The specified central frequencies of these stimulation channels were [ ]. As shown in FIG. 6A, the inter-pulse interval distributions for the six stimulation channels are centered on the specified central frequencies for these channels.

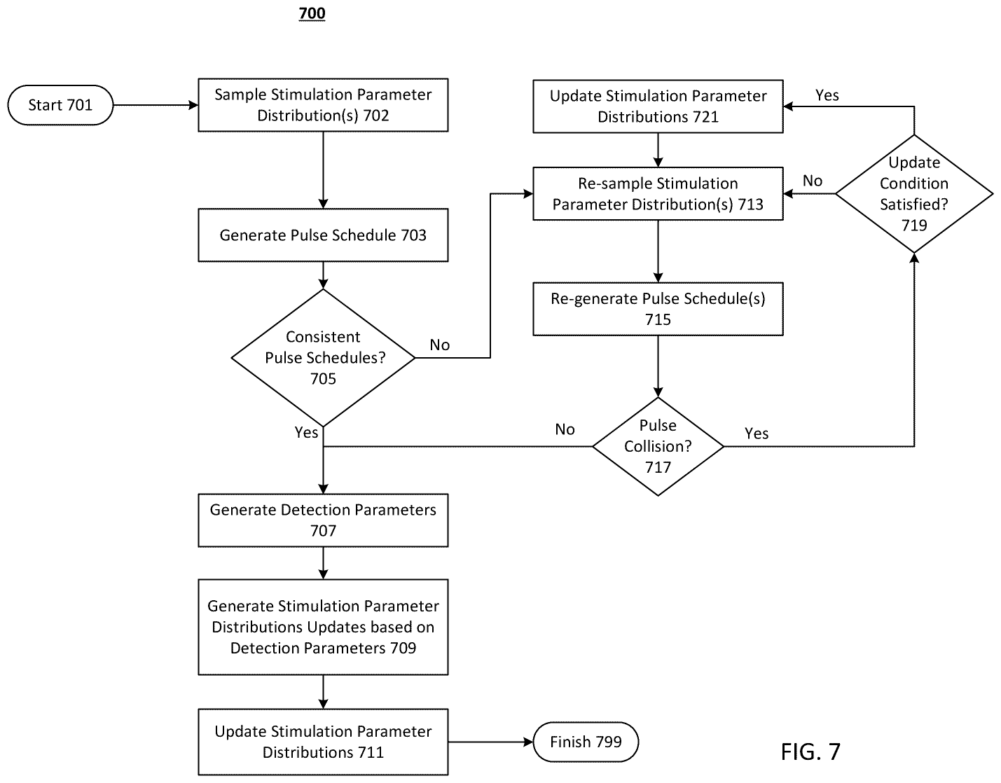

FIG. 7 depicts an exemplary process 700 of stochastic stimulation scheduling, consistent with disclosed embodiments. Process 700 can prevent pulse skipping or phase locking, while enabling the generation of consistent sets of stimulation pulses across multiple stimulation channels. In some embodiments, pulse schedule generation can be performed on a pulse-to-pulse basis, enabling on-the-fly addition or removal of stimulation channels without having to stop stimulation or determine whether stimulation channel parameters are consistent.

For convenience, the performance of process 700 is described with reference to an IPG (e.g., IPG 210) that implements the control signal generator and the controller described in FIG. 3. As may be appreciated, this description is not intended to be limiting. Process 700 can be performed using other control loop architectures. Furthermore, process 700 can be performed by an external controller (e.g., external controller 230) or a programmer (e.g., programmer 240).

In step 701, process 700 can start. In some embodiments, process 700 can start in response to the completion of a stimulation pulse. In various embodiments, process 700 can start in response to an elapsed time, according to a schedule, or in response to the modification of a stimulation partiture (e.g., the addition, removal, activation, deactivation, or modification of a stimulation channel).

In step 702 of process 700, the control signal generator can sample one or more stimulation parameter distributions associated with a stimulation channel to obtain corresponding sampled stimulation parameters.

In step 703 of process 700, the control signal generator can generate a pulse schedule using the sample stimulation parameters. In some instances, the control signal generator can also use additional stimulation parameters to generate the pulse schedule. These additional stimulation parameters can be specified parameters (e.g., through a programming process by a user), default parameters, or derived parameters. Derived parameters can be determined from combinations of other parameters (e.g., a stimulation amplitude can be derived from a sampled pulse duration using a strength duration curve, or vice versa).

In step 705 of process 700, the control signal generator can determine whether the generated pulse schedule is consistent with one or more other pulse schedules. As described herein, the control signal generator can determine whether pulse schedules are consistent according to rules, which can be specified rules (e.g., through a programming process by a user) or default rules. In some embodiments, pulse schedules can be inconsistent when they contain overlapping pulses (or, additionally, when a pulse in one pulse schedule ends within a predetermined time of the beginning of a pulse in another pulse schedule). If the pulse schedules are consistent, process 700 can proceed to step 707. Otherwise, process 700 can proceed to step 713.

In step 707 of process 700, the control signal generator can generate detection parameters, consistent with disclosed embodiments. In some embodiments, the detection parameters can include any suitable information usable to estimate the difficulty of generating sets of consistent pulse schedules. For example, the detection parameters can include an indication of the number of attempts required to generate the set of consistent stimulation parameters. In some embodiments, the detection parameters can include any suitable information usable to determine the fidelity of the generated pulse schedules. For example, the detection parameters can include an indication of the difference between the sampled parameters and any corresponding specified parameters (e.g., a jitter matrix).

In step 709 of process 700, the controller can generate stimulation parameter distribution updates based on the detection parameters received from the control signal generator. In some embodiments, the controller can estimate the difficulty of generating sets of consistent pulse schedules. For example, the controller can estimate parameter(s) of a difficulty distribution that describes the number of attempts required to generate a set of consistent pulse schedules. In some embodiments, the controller can determine error term(s) or sampled stimulation parameter distribution(s) that characterize the fidelity of the generated pulse schedules to specified pulse schedules.

Based on the estimated difficulty parameter(s) (or the estimated difficulty parameter(s) and estimated fidelity), the controller can determine updates to the stimulation parameter distributions. In some embodiments, the updates can depend on a difference between the estimated difficulty parameter(s) and target difficulty parameter(s). In some embodiments, the updates can be configured to reduce the error term(s) or shape the sampled stimulation parameter distribution(s) (e.g., by reducing the spread of the sampled stimulation parameter distribution(s)). In some embodiments, an update to a stimulation parameter distribution for a stimulation channel can depend on a specified value of the stimulation parameter for that stimulation channel. For example, an update to the variance of a stimulation parameter distribution (e.g., stimulation frequency or inter-pulse interval) for a stimulation channel can be weighted by the specified value of the mean for that stimulation parameter distribution.

In step 711 of process 700, the control signal generator can update the stimulation parameter distributions using the stimulation parameter distribution updates received from the controller. As may be appreciated, the disclosed embodiments encompass embodiments in which the controller, or another module or device, maintains the stimulation parameter distributions. In such embodiments, the controller (or the other module or device) can update the stimulation parameter distributions. The control signal generator can then obtain sampled stimulation parameter values from the controller, or other module or device.

In step 713 of process 700, in response to a determination that the pulse schedules are inconsistent, the control signal generator can re-sample the stimulation parameter distribution(s). In some embodiments, the control signal generator can re-sample the stimulation parameter distribution(s) associated with the stimulation channel of step 702. In various embodiments, the control signal generator can re-sample the stimulation parameter distribution(s) associated with all stimulation channels (or all non-protected stimulation channels).

In step 715 of process 700, the control signal generator can generate pulse schedule(s) using the sample stimulation parameter(s). In some instances, the control signal generator can also use additional stimulation parameters to generate the pulse schedule, as described with regards to step 703.

In step 717 of process 700, the control signal generator can determine whether the generated pulse schedule(s) are consistent, as described with regards to step 705. If the pulse schedules are consistent, process 700 can proceed to step 707. Otherwise, process 700 can proceed to step 719.

In step 719 of process 700, the control signal generator can determine whether an update condition is satisfied. In some embodiments, the update condition can be satisfied when the number of attempts exceeds a threshold value (e.g., 100 attempts, 1000 attempts, or a higher threshold). The threshold value can be selected to ensure that the control signal generator produced a timely new pulse schedule. It can be selected based on the speed at which the control signal generator can sample stimulation parameter distributions, generate new pulse schedules, and check pulse schedules for consistency. The faster the control signal generator can perform these functions, the higher the threshold value. When the update condition is satisfied, process 700 can proceed to step 721. Otherwise, process 700 can return to step 713 and the control signal generator can generate another set of pulse schedules.

In step 721 of process 700, in response to satisfaction of the update condition, the control signal generator can update the stimulation parameter distributions, consistent with disclosed embodiments. In some embodiments, the predetermined update can be or include an increase in the spreads of one or more stimulation parameter distributions. For example, when the stimulation parameter distributions are specified by means and variances, the predetermined update can be or include increasing the variance of at least one stimulation parameter distribution by between 10% and 1000%, or more. As an additional example, the predetermined update can be or include increasing the mean(s) of at least one stimulation parameter distribution by between 5% and 50%, or more.

In some embodiments, the control signal generator and controller can continue to manage the stimulation parameter distributions, following the update of the at least one stimulation parameter distribution. To continue the prior example, subsequent stimulation parameter distribution updates may be determined by the controller and applied to the variance increased in step 721, or the control signal generator can maintain stimulation parameter distributions having the updated means.

In some embodiments, the updates to pulse parameter distributions updated in step 721 can persist until a persistence condition is satisfied. For example, such updates can persist until a set of consistent pulse schedules is generated (or a number of sets of consistent pulse schedules are generated). As an additional example, such updates can persist for a certain time. Once the persistence condition is satisfied, the updates can be rolled back to the prior stimulation parameter distribution values (e.g., the prior means or variances).

In step 799, process 700 can finish. In some embodiments, performance of process 700 can result in the creation of a set of consistent pulse schedules (which may be used by a stimulator to provide stimulation to a patient) and in the updating of the stimulation parameter distributions.

As may be appreciated, process 700 includes a control loop. The beginning and ending of this control loop have been selected for ease of description and are not intended to be limiting. Other starting and ending points could alternatively be selected without departing from the envisioned embodiments. For example, the generation of the stimulation parameter distribution updates could be taken as the first step, and generation of the detection parameters taken as the last step.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware, but systems and methods consistent with the present disclosure can be implemented with hardware and software. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

The disclosed embodiments may further be described using the following clauses:
1. A stimulation scheduling method, comprising: sampling a stimulation parameter distribution for a first stimulation channel to obtain a stimulation parameter value; generating a first pulse schedule for the first stimulation channel using the stimulation parameter value; comparing the first pulse schedule to a second pulse schedule for a second stimulation channel; and based on the comparison, providing a stimulator control signal to a stimulator, the stimulator control signal based on the first pulse schedule and the stimulator configured to provide stimulation according to the stimulator control signal on the first stimulation channel.
2. The stimulation scheduling method of clause 1, the method further comprising: updating the stimulation parameter distribution based on a number of attempts required to generate the first pulse schedule.
3. The stimulation scheduling method of clause 2, wherein: updating the stimulation parameter distribution based on the number of attempts required to generate the first pulse schedule comprises: estimating a difficulty distribution parameter using the number of attempts required to generate the first pulse schedule; and updating the stimulation parameter distribution using the estimated difficulty distribution parameter.
4. The stimulation scheduling method of clause 2, wherein: updating the stimulation parameter distribution based on the number of attempts required to generate the first pulse schedule comprises: determining the number of attempts exceeds a threshold value; and in response to the determination, increasing a mean or spread of the stimulation parameter distribution.
5. The stimulation scheduling method of clause 2, wherein: updating the stimulation parameter distribution based on the number of attempts required to generate the first pulse schedule comprises: estimating a difficulty distribution parameter; and generating a control signal based on the difficulty distribution parameter.
6. The stimulation scheduling method of clause 5, wherein: updating the stimulation parameter distribution based on the number of attempts required to generate the first pulse schedule further comprises: weighting the control signal based on at least one specified stimulation parameter of the stimulation channel.
7. The stimulation scheduling method of any one of clauses 2 to 6, wherein: the stimulation parameter distribution comprises a stimulation parameter spread and updating the stimulation parameter distribution comprises updating the stimulation parameter spread.
8. The stimulation scheduling method of any one of clauses 1 to 7, the method further comprising: updating the stimulation parameter distribution using the first and second pulse schedules and specified inter-pulse intervals for the first and second stimulation channels.
9. The stimulation scheduling method of clause 8, wherein: updating the stimulation parameter distribution using the first and second pulse schedules and the specified inter-pulse intervals comprises: determining a jitter matrix based on respective differences between the first and second pulse schedules and the specified inter-pulse intervals for the first and second stimulation channels; and determining a pulse schedule fidelity using the jitter matrix.
10. The stimulation scheduling method of any one of clauses 1 to 9, wherein: the stimulation parameter value comprises an inter-pulse interval or a pulse phase duration.
11. The stimulation scheduling method of any one of clauses 1 to 9, wherein: the stimulation parameter value comprises a pulse phase duration; and generating the first pulse schedule using the stimulation parameter value comprises generating a pulse amplitude corresponding to the pulse phase duration.
12. A stimulation scheduling system, comprising: a control signal generator configured to: generate a set of consistent pulse schedules according to a set of corresponding stimulation parameter distributions; provide a stimulator control signal to a stimulator, the stimulator control signal based on the set of consistent pulse schedules and the stimulator configured to provide stimulation according to the stimulator control signal on a set of stimulation channels; and provide at least one detection parameter based on the generation of the set of consistent pulse schedules to a controller; and wherein the controller is configured to: estimate a difficulty state based on the at least one detection parameter; and update the stimulation parameter distributions based on the estimated difficulty state.
13. The stimulation scheduling system of clause 12, wherein: the stimulation parameter distributions comprise stimulation parameter spreads; and updating the stimulation parameter distributions comprises updating the stimulation parameter spreads.
14. The stimulation scheduling system of clause 13, wherein: the stimulation parameter distributions further comprise stimulation parameter means; and the updates to the stimulation parameter spreads depend upon: the estimated difficulty state; and the stimulation parameter means.
15. The stimulation scheduling system of clause 12, wherein: the stimulation parameter distributions comprise stimulation parameter means; and the controller is further configured to: determine an update condition is satisfied; and in response to the determination, update the stimulation characteristic means.
16. The stimulation scheduling system of any one of clauses 12 to 15, wherein: the at least one detection parameter comprises a number of attempts required to generate the consistent pulse schedules.
17. The stimulation scheduling system of any one of clauses 12 to 16, wherein: the estimated difficulty state comprises a parameter specifying an exponential or geometric distribution.
18. The stimulation scheduling system of any one of clauses 12 to 17, wherein: the control signal generator and the controller form a control loop configured to maintain a specified degree of difficulty in generating sets of consistent pulse schedules.
19. The stimulation scheduling system of any one of clauses 12 to 18, wherein: the stimulation parameter distributions comprise inter-pulse interval distributions or pulse phase duration distributions.
20. The stimulation scheduling system of any one of clauses 12 to 18, wherein: the stimulation parameter distributions comprise pulse phase duration distributions; and generating the set of pulse schedules comprises: sampling the pulse phase duration distributions to obtain pulse phase duration values; and generating pulse phase amplitudes corresponding to the pulse phase duration values.
21. A stimulation scheduling system, comprising: an implantable pulse generator configured to target, using a control signal generator and a controller, a degree of difficulty in generating consistent pulse schedules for a set of stimulation channels by updating stimulation parameter distributions for the stimulation channels, the stimulation parameter distributions used to generate the consistent pulse schedules.
21. The stimulation scheduling system of clause 20, wherein: the implantable pulse generator configured to is further configured to target a fidelity of the generated pulse schedules to specified stimulation parameters.
   Other embodiments will be apparent from consideration of the specification and practice of the embodiments disclosed herein. It is intended that the specification and examples be considered as example only, with a true scope and spirit of the disclosed embodiments being indicated by the following claims.

What is claimed:

1. A stimulation scheduling method, comprising:
sampling a stimulation parameter distribution for a first stimulation channel to obtain a stimulation parameter value;
generating a first pulse schedule for the first stimulation channel using the stimulation parameter value;
comparing the first pulse schedule to a second pulse schedule for a second stimulation channel; and
based on the comparison, providing a stimulator control signal to a stimulator, the stimulator control signal based on the first pulse schedule and the stimulator configured to provide stimulation according to the stimulator control signal on the first stimulation channel.

2. The stimulation scheduling method of claim 1, the method further comprising:
updating the stimulation parameter distribution based on a number of attempts required to generate the first pulse schedule.

3. The stimulation scheduling method of claim 2, wherein:
the stimulation parameter distribution comprises a stimulation parameter spread and updating the stimulation parameter distribution comprises updating the stimulation parameter spread.

4. The stimulation scheduling method of claim 2, wherein:
updating the stimulation parameter distribution based on the number of attempts required to generate the first pulse schedule comprises:
estimating a difficulty distribution parameter using the number of attempts required to generate the first pulse schedule; and
updating the stimulation parameter distribution using the estimated difficulty distribution parameter.

5. The stimulation scheduling method of claim 2, wherein:
updating the stimulation parameter distribution based on the number of attempts required to generate the first pulse schedule comprises:
determining the number of attempts exceeds a threshold value; and
in response to the determination, increasing a mean or spread of the stimulation parameter distribution.

6. The stimulation scheduling method of claim 2, wherein:
updating the stimulation parameter distribution based on the number of attempts required to generate the first pulse schedule comprises:
estimating a difficulty distribution parameter; and
generating a control signal based on the difficulty distribution parameter.

7. The stimulation scheduling method of claim 6, wherein:
updating the stimulation parameter distribution based on the number of attempts required to generate the first pulse schedule further comprises:
weighting the control signal based on at least one specified stimulation parameter of the stimulation channel.

8. The stimulation scheduling method of claim 1, the method further comprising:
updating the stimulation parameter distribution using the first and second pulse schedules and specified inter-pulse intervals for the first and second stimulation channels.

9. The stimulation scheduling method of claim 8, wherein:
updating the stimulation parameter distribution using the first and second pulse schedules and the specified inter-pulse intervals comprises:
determining a jitter matrix based on respective differences between the first and second pulse schedules and the specified inter-pulse intervals for the first and second stimulation channels; and
determining a pulse schedule fidelity using the jitter matrix.

10. The stimulation scheduling method of claim 1, wherein:
the stimulation parameter value comprises an inter-pulse interval or a pulse phase duration.

11. The stimulation scheduling method of claim 1, wherein:
the stimulation parameter value comprises a pulse phase duration; and
generating the first pulse schedule using the stimulation parameter value comprises generating a pulse amplitude corresponding to the pulse phase duration.

12. A stimulation scheduling system, comprising:
a control signal generator configured to:
generate a set of consistent pulse schedules according to a set of corresponding stimulation parameter distributions;
provide a stimulator control signal to a stimulator, the stimulator control signal based on the set of consistent pulse schedules and the stimulator configured to provide stimulation according to the stimulator control signal on a set of stimulation channels; and
provide at least one detection parameter based on the generation of the set of consistent pulse schedules to a controller; and
wherein the controller is configured to:
estimate a difficulty state based on the at least one detection parameter; and
update the stimulation parameter distributions based on the estimated difficulty state.

13. The stimulation scheduling system of claim 12, wherein:
the stimulation parameter distributions comprise stimulation parameter spreads; and
updating the stimulation parameter distributions comprises updating the stimulation parameter spreads.

14. The stimulation scheduling system of claim 13, wherein:
the stimulation parameter distributions further comprise stimulation parameter means; and
the updates to the stimulation parameter spreads depend upon:
the estimated difficulty state; and
the stimulation parameter means.

15. The stimulation scheduling system of claim 12, wherein:
the stimulation parameter distributions comprise stimulation parameter means; and
the controller is further configured to:
determine an update condition is satisfied; and
in response to the determination, update the stimulation characteristic means.

16. The stimulation scheduling system of claim 12, wherein:
the at least one detection parameter comprises a number of attempts required to generate the consistent pulse schedules.

17. The stimulation scheduling system of claim 12, wherein:
the estimated difficulty state comprises a parameter specifying an exponential or geometric distribution.

18. The stimulation scheduling system of claim 12, wherein:
the control signal generator and the controller form a control loop configured to maintain a specified degree of difficulty in generating sets of consistent pulse schedules.

19. The stimulation scheduling system of claim 12, wherein:
the stimulation parameter distributions comprise inter-pulse interval distributions or pulse phase duration distributions.

20. The stimulation scheduling system of claim 12, wherein:
the stimulation parameter distributions comprise pulse phase duration distributions; and
generating the set of pulse schedules comprises:
sampling the pulse phase duration distributions to obtain pulse phase duration values; and
generating pulse phase amplitudes corresponding to the pulse phase duration values.

21. A stimulation scheduling system, comprising:
an implantable pulse generator configured to target, using a control signal generator and a controller, a degree of difficulty in generating consistent pulse schedules for a set of stimulation channels by updating stimulation parameter distributions for the stimulation channels, the stimulation parameter distributions used to generate the consistent pulse schedules.

22. The stimulation scheduling system of claim 21, wherein:
the implantable pulse generator configured to is further configured to target a fidelity of the generated pulse schedules to specified stimulation parameters.
